(19) United States
(12) Patent Application Publication
(10) Pub. No.: US 2009/0029450 A1
(43) Pub. Date: Jan. 29, 2009

(54) Incubator

(75) Inventors: Kyoji Nakamura, Kanagawa-ken (JP); Takayuki Uozumi, Tokyo (JP)

Correspondence Address: OLIFF & BERRIDGE, PLC, P.O. BOX 320850, ALEXANDRIA, VA, 22320-4850, US

(73) Assignee: NIKON CORPORATION, TOKYO (JP)

(21) Appl. No.: 11/878,546

(22) Filed: Jul. 25, 2007

(51) Int. Cl. C12M 1/36

(52) U.S. Cl. 435/286.2

(57) ABSTRACT

An accommodating unit capable of accommodating a culture vessel, a transfer unit supporting and transferring the culture vessel, and taking in and out the culture vessel relative to the accommodating unit, and an observation unit observing a sample inside of the culture vessel via an observation optical system under a state in which the culture vessel is supported by the transfer unit, are included. The transfer unit performs a positioning of each part of the sample on an optical axis of the observation optical system by moving the culture vessel at the time of observation of the sample by the observation unit. Consequently, it is possible to provide an incubator having a simple configuration.

### BACKGROUND

1. Field

The present invention relates to an incubator performing a culture of a sample.

2. Description of the Related Art

An incubator is known as a device culturing a sample such as cells (for example, refer to Japanese Unexamined Patent Application Publication No. 2004-180675). Besides, it is proposed that a microscopic observation device is provided inside of a housing of the incubator to observe a state of the sample during culturing by this microscopic observation device. It is not necessary to take the sample out of the housing for observation in the incubator as stated above, and it is possible to perform the observation of the sample inside of the housing (namely, while keeping a stable culture environment).

Further, in the above-stated incubator, a culture vessel of the sample is transferred by a transfer system inside of the housing to place the culture vessel on a stage of the microscopic observation device before the sample is observed. When the sample inside of the culture vessel on the stage is observed, each part of the sample is positioned on an optical axis of the microscopic observation device by moving an optical system, a camera, and so on of the microscopic observation device finely by a stage for observation provided in addition to the transfer system.

However, the above-stated incubator has a complicated configuration because it has the stage for observation in addition to the transfer system of the culture vessel.

### SUMMARY

An object of the present invention is to provide an incubator having a simple configuration.

An aspect of the present invention includes: an accommodating unit capable of accommodating a culture vessel; a transfer unit supporting and transferring the culture vessel, and taking in and out the culture vessel relative to the accommodating unit; and an observation unit observing a sample inside of the culture vessel via an observation optical system under a state in which the culture vessel is supported by the transfer unit. The transfer unit performs a positioning of each part of the sample on an optical axis of the observation optical system by moving the culture vessel when the sample is observed by the observation unit. Consequently, it becomes possible to provide an incubator having a simple configuration.

It is preferable that the transfer unit transfers the culture vessel at least in a direction in parallel with the optical axis before the observation of the sample by the observation unit, and stops the transfer of the culture vessel in the parallel direction when the sample reaches in a vicinity of a focal surface of the observation unit, and the observation unit performs a focusing of the sample by moving a part of an image formation optical system forming an image of the sample in the parallel direction, after the transfer of the culture vessel in the parallel direction is stopped.

Besides, it is preferable that the accommodating unit and the transfer unit are provided inside of a housing maintaining a culture environment of the sample, the observation unit is provided outside of the housing, transparent window parts for observation are provided at portions facing the observation unit at the housing, and a vicinity of the window parts of the housing becomes a space where the transfer unit transfers the culture vessel at the time of observation of the sample by the observation unit.

Besides, it is preferable that a transparent window part for visual observation is provided at the housing.

Besides, it is preferable that the window part is composed of a heater glass.

Besides, it is preferable that a projection part projecting toward outside is provided at the housing, and the window parts and the space are provided at the projection part.

### BRIEF DESCRIPTION OF THE DRAWINGS

The nature, principle, and utility will become more apparent from the following detailed description when read in conjunction with the accompanying drawings in which like parts are designated by identical reference numbers, in which:

FIG. 1 is a front view showing an entire configuration of an incubator;

FIG. 2 is an upper surface view of FIG. 1;

FIG. 3 is a view showing a state in which a rotary table of an automatic transfer unit rotates 180 degrees;

FIG. 4 is a front view showing a state in which a holder is transferred to a space inside of a projection part by the automatic transfer unit;

FIG. 5 is an upper surface view of FIG. 4; and

FIG. 6 is an enlarged view showing each configuration of an illumination part and an observation part of a microscope unit.

### DETAILED DESCRIPTION OF THE EMBODIMENTS

Hereinafter, embodiments of the present invention are described in detail with reference to the drawings.

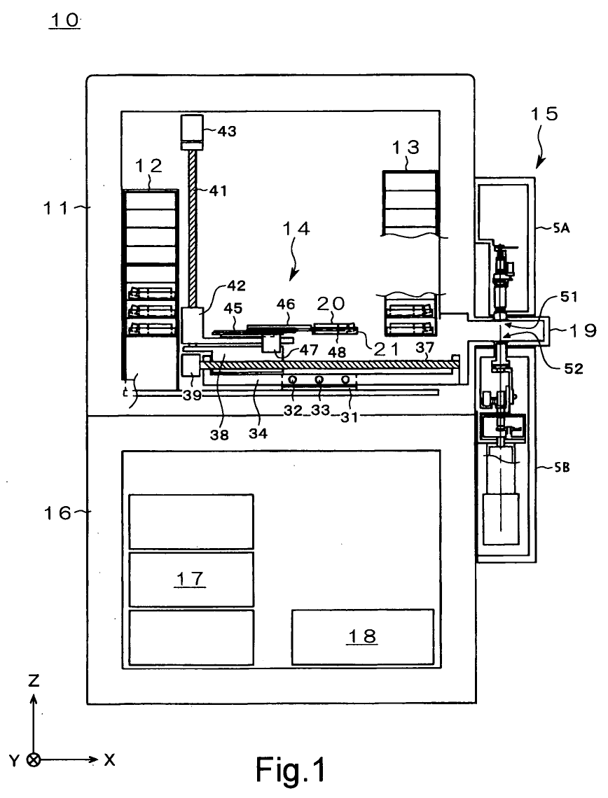

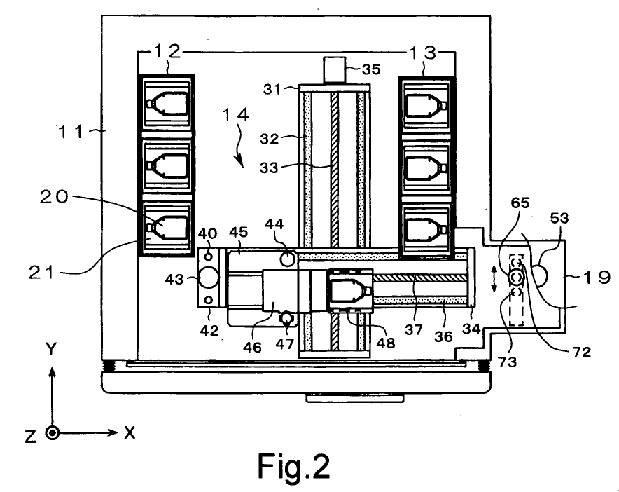

An incubator 10 of the present embodiment is constituted by a housing 11, stockers 12, 13, an automatic transfer unit 14 and a microscope unit 15 as shown in FIG. 1 and FIG. 2. Among them, the stockers 12, 13 and the automatic transfer unit 14 are provided inside of the housing 11, and the microscope unit 15 is provided outside (lateral side) of the housing 11. Besides, a housing 16 is provided at downward of the housing 11, and a control box 17, a personal computer 18, and so on are provided inside thereof.

A culture of a sample (not shown) such as cells is performed inside of the housing 11. Inside of the housing 11 is sealed so as to maintain a culture environment at the sample during culturing of the sample, and is kept at a constant temperature by, for example, circulating air. Besides, a projection part 19 projecting toward outside is provided at a side surface of the housing 11. A space inside of the projection part 19 is also kept at the constant temperature in accordance with the culture environment of the sample. The housing 11 is composed of, for example, a stainless steel material. The sample such as the cells is placed on a holder 21 under a state injected in a culture vessel 20.

One or more culture vessels 20 together with the holders 21 can be accommodated in the stockers 12, 13. Respective steps of the stockers 12, 13 may be constituted corresponding to the individual holders 21, or the respective steps of the stockers 12, 13 may be constituted to suite to a size of a carry gathering the plural holders 21 together. Besides, the stockers 12, 13 are provided along side walls inside of the housing 11.

The automatic transfer unit 14 is a mechanism to support and transfer the holder 21, and to take in and out the holder 21 relative to the stockers 12, 13. Incidentally, before the culture of the sample is started, the individual holders 21 are passed to the automatic transfer unit 14 inside of the housing 11 by a not-shown automatic carry-in mechanism provided outside of the housing 11, to be accommodated in the respective steps of the stockers 12, 13.

Here, a schematic configuration of the automatic transfer unit 14 is described.

A Y stage 34 is attached to a stage base 31 via a guide shaft 32 and a drive shaft 33 for Y axis. The Y stage 34 moves in the Y axis direction by a rotation of a motor 35. Incidentally, the stage base 31 is fixed relative to the housing 11.

An X stage 38 is attached to the Y stage 34 via a guide shaft 36 and a drive shaft 37 for X axis. The X stage 38 moves in the X axis direction by a rotation of a motor 39.

A Z stage 42 is attached to the X stage 38 via a guide shaft 40 and a drive shaft 41 for Z axis. The Z stage 42 moves in the Z axis direction by a rotation of a motor 43.

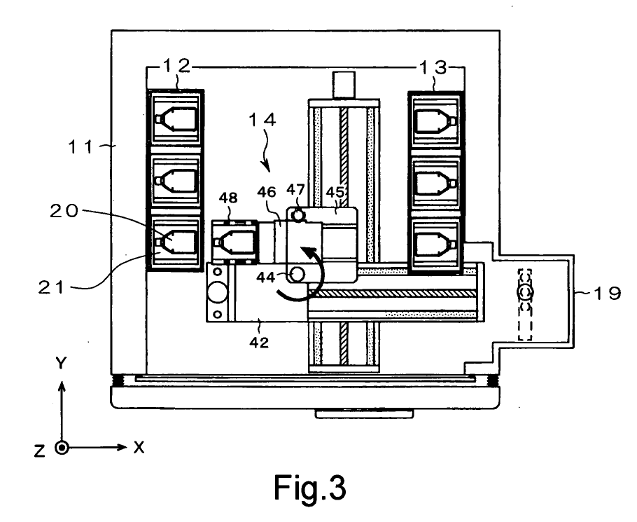

A rotation stage 45 is attached to the Z stage 42 via a rotation shaft 44. The rotation shaft 44 is in parallel with the Z axis. The rotation stage 45 rotates within an XY surface by a rotation of a not-shown motor. When the rotation stage 45 is rotated 180 degrees, a state changes from FIG. 2 to FIG. 3.

A mini stage 46 is attached to the rotation stage 45 via a not-shown rack and pinion. The mini stage 46 is a stage provided at an uppermost part of the automatic transfer unit 14, and moves in the X axis direction by a rotation of a motor 47 with small strokes.

An arm part 48 (shown by a dotted line in FIG. 2) is detachably attached at a tip portion of the mini stage 46. A collar portion of the holder 21 is placed on the arm part 48. The arm part 48 is used as a delivery member at the time of the transfer and setting of the holder 21.

In the above-stated automatic transfer unit 14, the holder 21 supported by the arm part 48 can be moved in the Y axis, X axis and Z axis directions, can be rotated 180 degrees centering on the Z axis, and further, can be moved (send out) with the small strokes in the X axis direction. Incidentally, when the holder 21 is rotated by the rotation stage 45, it is preferable that a radius of rotation is made small by shortening the mini stage 46 toward the rotation stage 45 side.

When the holder 21 is taken in and out to/from the stocker 12, the rotation stage 45 is rotated 180 degrees as shown in FIG. 3 and the arm part 48 is moved in the Y axis, X axis and Z axis directions in the automatic transfer unit 14. Besides, when the holder 21 is taken in and out to/from the stocker 13, the arm part 48 is moved in the Y axis, X axis and Z axis directions without rotating the rotation stage 45 (a state in FIG. 2).

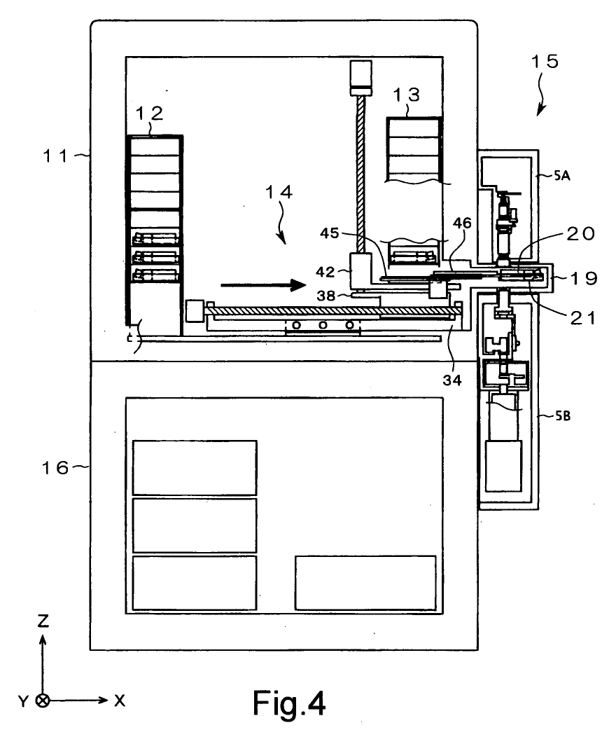

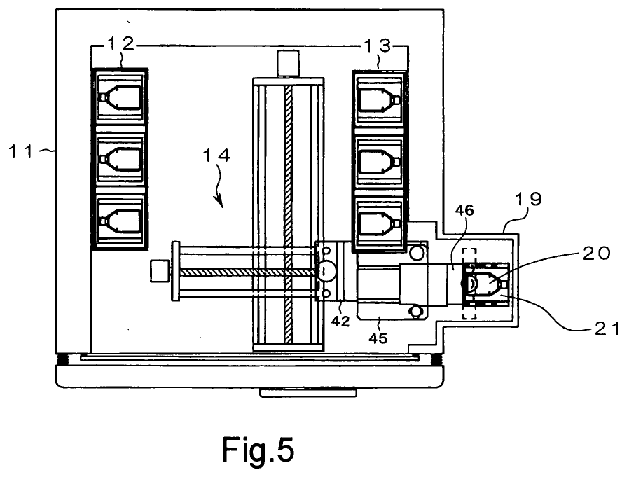

Further, the guide shaft 36 and the drive shaft 37 for X axis are extended in a vicinity of the projection part 19 of the housing 11 in the automatic transfer unit 14. Accordingly, a movable part from the X stage 38 to the mini stage 46 is moved in the vicinity of the projection part 19 (states in FIG. 4 and FIG. 5), and thereby, it becomes possible to transfer the holder 21 supported by the arm part 48 to the space inside of the projection part 19. The transfer as stated above is performed when a state of the sample during culturing is observed by the microscope unit 15.

The microscope unit 15 is provided outside (lateral side) of the housing 11, and provided separately above and below so as to sandwich the projection part 19. An upper side of the microscope unit 15 is an illumination system 5A, and a lower side thereof is an observation system 5B. When the holder 21 is transferred to the space inside of the projection part 19, the sample inside of the culture vessel 20 on the holder 21 is disposed on the optical axis of the illumination system 5A, observation system 5B of the microscope unit 15.

Incidentally, a transparent window part 51 for observation is provided at an upper side portion facing the illumination system 5A, and a similar transparent window part 52 is provided at a lower side portion facing the observation system 5B at the projection part 19 of the housing 11, as shown in FIG. 1. Besides, a transparent window part 53 (FIG. 2) for visual observation is also provided in a vicinity of the window part 51. These window parts 51 to 53 are composed of glass heaters, and dew condensation can be prevented. The glass heater is the one in which a heater in a sheet state is pasted on a glass plate.

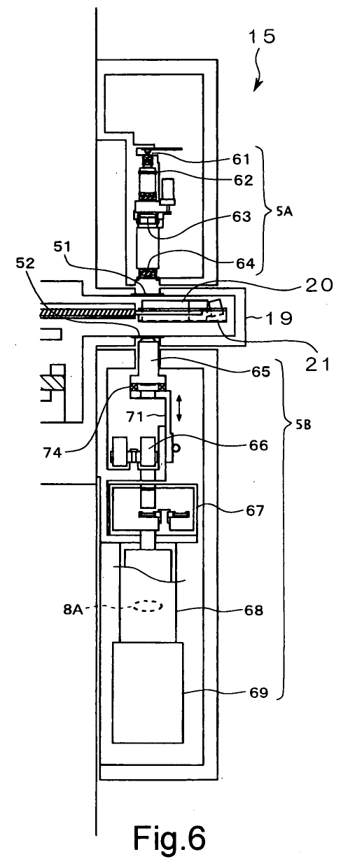

The space in the vicinity of the window parts 51 to 53 (the space inside of the projection part 19 in the present embodiment) is the space to which the automatic transfer unit 14 transfers the holder 21 at the time of the observation of the sample by the microscope unit 15. The microscope unit 15 performs the observation of the sample inside of the culture vessel 20 under a state in which the holder 21 is supported by the arm part 48 of the automatic transfer unit 14. The illumination system 5A and the observation system 5B of the microscope unit 15 are described with reference to FIG. 6.

In the illumination system 5A, light from a light source 61 such as an LED is incident on the window part 51 of the projection part 19 of the housing 11 after passing through a rectangular aperture 62, a phase ring 63 and a condenser lens 64. The light passing through the window part 51 is incident on the sample inside of the culture vessel 20 of the holder 21 transferred to the space inside of the projection part 19, as illumination light. The sample illuminated by the light from the illumination system 5A generates light in accordance with the culture state. The light generated from the sample in a transmission direction is guided to the observation system 5B after passing through the window part 52 of the projection part 19.

In the observation system 5B, the light generated from the sample in the transmission direction is incident on a CCD camera 69 after passing through an objective lens 65, an intermediate variable lens 66, a fluorescent lighting unit 67, and a built-in lens 8A of a CCD supporting table 68. At this time, an image of the sample by an image formation optical system (65, 66, 8A) is formed on an image forming surface of the CCD camera 69.

Besides, an up and down mechanism 71 moving the objective lens 65 in a direction parallel to the optical axis (hereinafter, called as a “Z direction”) is provided at the observation system 5B. The objective lens 65 is horizontally moved by the up and down mechanism 71, and thereby, a focusing (focus adjustment) of the sample can be performed. Further, objective lenses 72, 73 (refer to FIG. 2) having different magnifications are disposed at a periphery of the objective lens 65, and they can be switched by a guide rail 74 (refer to FIG. 6). Besides, the plural intermediate variable lenses 66 are prepared, and they can be switched by an electric turret.

When the sample during culturing is observed by using the microscope unit 15 having the above-stated configuration, the automatic transfer unit 14 takes out the holder 21 from the stockers 12, 13, and transfers to the space in the projection part 19 of the housing 11 under the state supported by the arm part 48 (refer to FIG. 4, FIG. 5). The observation can be performed without damaging the sample during culturing because the above-stated space is also kept in the constant temperature in accordance with the culture environment of the sample.

The holder 21 is transferred by the automatic transfer unit 14 also in the projection part 19 of the housing 11, and each part of the sample inside of the culture vessel 20 on the holder 21 is sequentially positioned on the optical axis of the microscope unit 15. Consequently, it becomes possible to observe each part of the sample and confirm the culture state of the sample based on images outputted from the CCD camera 69 to, for example, a monitor device and so on while keeping the stable culture environment.

As stated above, in the incubator 10 of the present embodiment, each part of the sample is observed while transferring the holder 21 under the state in which the holder 21 is supported by the arm part 48 of the automatic transfer unit 14, namely, each part of the sample is observed by using the automatic transfer unit 14, and therefore, it is not necessary to provide a stage for observation in addition to the automatic transfer unit 14 (for example, a mechanism moving the objective lens 65 and so on of the observation system 5B within a surface perpendicular to the optical axis).

Consequently, it is possible to obtain the incubator 10 having a simple configuration. The incubator 10 as stated above is suitable for a culture of a sample in a regenerative medicine, a development of new drugs field, and so on. Incidentally, a positioning control of the holder 21 in an XY direction is performed under the state in which the holder 21 is supported by the arm part 48 of the automatic transfer unit 14, and therefore, it is preferable that a member having a large friction resistance (for example, rubber and so on) is pasted on a contact surface of the arm part 48 and the holder 21.

Besides, in the incubator 10 of the present embodiment, it is preferable that a transfer control and an AF control as stated below are to be performed before the observation of the sample by the microscope unit 15. Namely, the holder 21 is taken out of the stockers 12, 13 to transfer at least in the Z direction, and when the sample reaches in a vicinity of a focal surface of the microscope unit 15, the transfer of the holder 21 in the Z direction is stopped, and thereafter, the transfer control is performed while limiting within an XY surface in the vicinity of the focal surface.

On the other hand, in the microscope unit 15, after the transfer of the holder 21 in the Z direction by the automatic transfer unit 14 is stopped, the objective lens 65 of the observation system 5B is moved in the Z direction to perform the focusing of the sample. The AF control as stated above is preferable to be performed every time when each part of the sample is positioned on the optical axis of the microscope unit 15.

The transfer control and the AF control as stated above are performed, and thereby, it becomes possible to perform the positioning of the sample at the focal surface of the microscope unit 15 more preciously compared to the case when the focusing of the sample is performed by transferring the holder 21 in the Z direction by the automatic transfer unit 14 (the objective lens 65 is fixed). Accordingly, the observation of each part of the sample can be performed finely.

Further, in the incubator 10 of the present embodiment, the stockers 12, 13 and the automatic transfer unit 14 are provided inside of the housing 11, the microscope unit 15 is provided outside of the housing, and each part of the sample is observed via the window parts 51, 52 of the housing 11. Accordingly, the observation of the sample can be performed while keeping the stable culture environment without damaging the sample during culturing.

Besides, inside of the housing 11 is kept in high humid environment suitable for the culture of the sample, and therefore, there is a possibility that each optical component and so on constituting the microscope unit 15 may be damaged by the humidity, if the microscope unit 15 is provided inside thereof. However, in the incubator 10 of the present embodiment, the microscope unit 15 is provided outside of the housing 11, and therefore, the damage of each optical component caused by the humidity (for example, shortening of life span) can be surely avoided. In addition, there is also an advantage that a maintenance check of the microscope unit 15 becomes easy.

Further, the space inside of the housing 11 of the incubator 10 is limited, and therefore, there is awkwardness in providing the stockers 12, 13, the automatic transfer unit 14, and the microscope unit 15 within this space. If all of them are provided inside of the housing 11, it is not preferable because the housing 11 becomes large. In the incubator 10 of the present embodiment, the microscope unit 15 is provided outside of the housing 11, and therefore, the stockers 12, 13 and the automatic transfer unit 14 can be efficiently disposed within the limited space inside of the housing 11 while avoiding the enlargement of the housing 11.

Besides, in the incubator 10 of the present embodiment, the window part 53 for visual observation (FIG. 2) is provided in the vicinity of the window part 51 of the projection part 19 of the housing 11 (at a portion slightly out of an observation optical path). Accordingly, the holder 21 supported by the arm part 48 of the automatic transfer unit 14 and the culture vessel 20 on the holder 21 can be observed visually from outside.

Further, in the incubator 10 of the present embodiment, the projection part 19 is provided at the housing 11, the holder 21 is transferred to the space inside of the projection part 19, and each part of the sample of the holder 21 is observed via the window parts 51, 52 of the projection part 19. Accordingly, the microscopic observation of the sample can be performed easily. Besides, the projection part 19 can be used when the microscope unit 15 is provided outside of the housing 11, and the setting thereof can be performed easily.

### Modified Example

Incidentally, in the above-stated embodiment, the objective lens 65 of the observation system 5B is moved in the Z direction to perform the focusing of the sample, but the present invention is not limited to the above. The objective lens 65 may be fixed and the holder 21 may be transferred in the Z direction by the automatic transfer unit 14 to perform the focusing of the sample.

Further, in the above-stated embodiment, the window parts 51 to 53 are provided at the projection part 19 of the housing 11, but the present invention is not limited to the above. When the visual observations of the holder 21, the culture vessel 20, and so on are not necessary, the window part 53 may not be provided. Besides, the projection part 19 may be constituted by a totally transparent member, without being limited to the constitution in which the transparent window parts 51 to 53 are provided at the opaque projection part 19.

Besides, an example in which the projection part 19 is provided at the housing 11 is explained in the above-stated embodiment, but the present invention is not limited to the above. A transparent member similar to the above (window part) may be provided at a predetermined portion (a portion facing the microscope unit 15) of the housing 11, and the holder 21 is transferred to a space in a vicinity of the window part, then it becomes possible to observe each part of a sample via this window part, even when the projection part 19 is not provided.

Further, in the above-stated embodiment, the microscope unit 15 is provided outside of the housing 11, but the present invention is not limited to the above. The microscope unit 15 may be provided inside of the housing 11 as same as the automatic transfer unit 14 and so on. However, it is preferable to shut out negative effects on each optical component caused by surrounding environment (a culture environment of a sample) by providing the microscope unit 15 inside of the housing 11 under a state accommodated in a housing having a sealed structure. In this case, window parts of the housing 11 become unnecessary.

Besides, in the above-stated embodiment, an example in which the collar portion of the holder 21 is placed on the arm part 48 when the holder 21 is supported by the arm part 48 of the automatic transfer unit 14 is explained, but the present invention is not limited to the above. In addition to the above, the holder 21 may be supported by a method sandwiching from lateral sides, or the holder 21 may be completely fixed to be held by using a pneumatic chuck, a magnet, and so on.

Further, the configuration of the automatic transfer unit 14 is not limited to the above-stated examples, but a drive operation function may be included so as to enable a manual observation. For example, it is conceivable that the arm part 48 of the automatic transfer unit 14 is moved in the Y axis, X axis, and Z axis directions in accordance with an input operation of a keyboard and so on of the personal computer 18. Besides, a handling mechanism to hold the culture vessel 20 may be exchangeably attached in the automatic transfer unit 14.

The many features and advantages of the embodiments are apparent from the detailed specification and, thus, it is intended by the appended claims to cover all such features and advantages of the embodiments that fall within the true spirit and scope thereof. Further, since numerous modifications and changes will readily occur to those skilled in the art, it is not desired to limit the inventive embodiments to the exact construction and operation illustrated and described, and accordingly all suitable modifications and equivalents may be resorted to, falling within the scope thereof.

What is claimed is:

1. An incubator comprising:
an accommodating unit capable of accommodating a culture vessel;
a transfer unit supporting and transferring said culture vessel, and taking in and out said culture vessel relative to said accommodating unit; and
an observation unit observing a sample inside of the culture vessel via an observation optical system under a state in which said culture vessel is supported by said transfer unit and wherein
said transfer unit performs a positioning of each part of said sample on an optical axis of said observation optical system by moving said culture vessel when the sample is observed by said observation unit.

2. The incubator according to claim 1, wherein
said transfer unit transfers said culture vessel at least in a direction in parallel with said optical axis before the observation of said sample by said observation unit, and stops the transfer of said culture vessel in said parallel direction when said sample reaches in a vicinity of a focal surface of said observation unit and
said observation unit performs a focusing of said sample by moving a part of an image formation optical system forming an image of said sample in said parallel direction, after the transfer of said culture vessel in said parallel direction is stopped.

3. The incubator according to claim 1, wherein
said accommodating unit and said transfer unit are provided inside of a housing maintaining a culture environment of said sample,
said observation unit is provided outside of said housing,
transparent window parts for observation are provided at portions of said housing facing said observation unit, and
a vicinity of said window parts of said housing becomes a space where said transfer unit transfers said culture vessel at the time of observation of said sample by said observation unit.

4. The incubator according to claim 3, wherein
a transparent window part for visual observation is provided at said housing.

5. The incubator according to claim 3, wherein
said window parts are composed of heater glasses.

6. The incubator according to claim 3, wherein
said housing provides a projection part projecting towards an outside, and said window parts and said space are provided at the projection part.
